(19) United States
(12) Patent Application Publication
(10) Pub. No.: US 2004/0198773 A1
(43) Pub. Date: Oct. 7, 2004

(54) Substituted 3-pyridyl oxazoles as c17,20 lyase inhibitors

(76) Inventors: Barry Hart, Palo Alto, CA (US); Donald E Bierer, Bethany, CT (US); Chengzhi Zhang, Orange, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/491,117

(22) PCT Filed: Sep. 26, 2002
(86) PCT No.: PCT/US02/30834

Related U.S. Application Data
(60) Provisional application No. 60/324,993, filed on Sep. 26, 2001.

(51) Int. Cl. C07D 413/04; C07D 263/30; A61K 31/4439; A61K 31/421

(52) U.S. Cl. 514/340; 514/374; 546/274.1; 548/235

(57) ABSTRACT

Substituted 3-pyridyl oxazoles which inhibit C_{17, 20} Lyase, pharmaceutical preparations containing them, and methods of using them in treatment of cancer arc provided.

### BACKGROUND OF THE INVENTION

Steroid biosynthesis begins in cells of the adrenal gland where the initial product in sterol biosynthesis, cholesterol, is converted into the adrenal steroid hormones aldosterone, hydrocortisone, and corticosterone by a series of P₄₅₀-mediated hydroxylation steps. The cholesterol side-chain cleavage activity that represents the first step in steroid hormone biosynthesis is a P₄₅₀ -mediated oxidation and cleavage of a pair of adjacent methylene groups to two carbonyl fragments, pregnenolone and isocaprylaldehyde (see Walsh (1979) Enzymatic Reaction Mechanisms; W. H. Freeman and Company, pp. 474-77). Another critical set of enzymatic conversions in steroid metabolism is facilitated by 17-alpha-hydroxylase-17,20lyase (CYP17, P₄₅₀ 17). CYP 17 is a bifunctional enzyme which possesses both a C17,20-lyase activity and a C17-hydroxylase activity. Significantly, these two alternative enzymatic activities of CYP17 result in the formation of critically different intermediates in steroid biosynthesis and each activity appear to be differentially and developmentally regulated (see e.g. l'Allemand et al. (2000) Eur. J. Clin. Invest. 30: 28-33).

The C17,20-lyase activity of CYP17 catalyzes the conversion of 17α-hydroxy-pregnenolone and 17α-hydroxy-progesterone to dehydroepiandrosterone (DHEA) and delta4-androstenedione (androstenedione) respectively. Both DHEA and androstenedione lyase products are key intermediates in the synthesis of not only the androgens testosterone and dihydrotestosterone (DHT), but also the estrogens 17-beta-estradiol and estrone. Indeed, adrenal and ovarian estrogens are the main sources of estrogens in postmenopausal women (see e.g. Harris et al. (1988) Br. J. Cancer 58: 493-6). In contrast, the C17-hydroxylase activity of CYP17 catalyzes the conversion of the common intermediate progesterone to 17-hydroxyprogesterone, a precursor of cortisol. Therefore the first activity of CYP17, the C17-hydroxylase activity, promotes the formation of glucocorticoids while the second activity of CYP 17, the C17,20-lyase activity, promotes the formation of sex hormones—particularly androgens including testosterone as well as estrogens.

Prostate cancer is currently one of the most frequently diagnosed forms of cancer in men in the U.S. and Europe. Prostate cancer is typically androgen-dependent and, accordingly, the reduction in androgen production via surgical or pharmacological castration remains the major treatment option for this indication. However, complete rather than partial withdrawal of androgens may be more effective in treating prostate cancer (Labrie, F. et al., Prostate, 1983, 4, 579 and Crawford, E. D. et al., N. Engl. J. Med., 1989, 321, 419). Pharmacological inhibition of CYP17 maybe a promising alternative treatment to antiandrogens and LHRH agonists in that testicular, adrenal, and peripheral androgen biosynthesis would be reduced rather than only testicular androgen production (Njar V, et al., J. Med. Chem., 1998, 41, 902). One such CYP17 inhibitor, the fungicide ketoconazole, has been used previously for prostate cancer treatment (Trachtenberg, J., J. Urol., 1984, 132, 61 and Williams, G. et al., Br. J. Urol., 1986, 58, 45). However, this drug is a relatively non-selective inhibitor of cytochrome P450 (CYP) enzymes, has weak CYP17 activity, and has a number of notable side effects associated with it including liver damage(De Coster, R. et al., J. Steroid Biochem. Mol. Biol., 1996, 56, 133 and Lake-Bakaar, G. et al., Br. Med. J., 1987, 294, 419).

The importance of potent and selective inhibitors of CYP17 as potential prostate cancer treatments has been the subject of numerous studies and reviews (Njar, V. et al., Curr. Pharm. Design, 1999, 5, 163; Barrie, S. E. et al., Endocr. Relat. Cancer, 1996, 3, 25 and Jarman, M. et al., Nat. Prod. Rep., 1998, 495). Finasteride, a 5α-reductase inhibitor, is an approved treatment for benign prostatic hyperplasia (BPH), although it is only effective with patients exhibiting minimal disease. While finasteride reduces serum DHT levels, it increases testosterone levels, and may therefore be insufficient for prostate cancer treatment (Peters, D. H. et al., Drugs, 1993, 46, 177). Certain anti-androgenic steroids, for example, cyproterone acetate (17α-acetoxy-6-chloro-1α, 2α-methylene4,6-pregnadiene-3,20-dione), have been tested as adjuvant treatments for prostate cancer. Many other steroids have been tested as hydroxylase/lyase inhibitors. See, for example, PCT Specification WO 92/00992 (Schering AG) which describes anti-androgenic steroids having a pyrazole or triazole ring fused to the A ring at the 2,3-position, or European specifications EP-A288053 and EP-A413270 (Merrell Dow) which propose 17β-cyclopropylamino-androst-5-en-3β-ol or -4-en-3-one and their derivatives.

In addition to the use of CYP17 inhibitors in the treatment of prostate cancer, a second potential indication would be for estrogen-dependent breast cancer. In postmenopausal patients with advanced breast cancer, treatment with high doses of ketoconazole resulted in suppression of both testosterone and estradiol levels, implicating CYP17 as a potential target for hormone therapy (Harris, A. L. et al., Br. J. Cancer, 1988, 58, 493).

Chemotherapy is usually not highly effective, and is not a practical option for most patients with prostate cancer because of the adverse side effects which are particularly detrimental in older patients. However, the majority of patients initially respond to hormone ablative therapy although they eventually relapse, as is typical with all cancer treatments (McGuire, in: Hornones and Cancer,. Iacobelli et al. Eds.; Raven Press, New York, 1980, Vol. 15, 337-344). Current treatment by orchidectomy or administration of gonadotropin-releasing hormone (GnRH) agonists results in reduced androgen production by the testis, but does not interfere with androgen synthesis by the adrenals. Following three months of treatment with a GnRH agonist, testosterone and DHT concentrations in the prostate remained at 25% and 10%, respectively, of pretreatment levels (Forti et al., J. Clin. Endocrinol. Metab., 1989, 68, 461). Similarly, about 20% of castrated patients in relapse had significant levels of DHT in their prostatic tissue (Geller et al., J. Urol., 1984, 132, 693). These findings suggest that the adrenals contribute precursor androgens to the prostate. This is supported by clinical studies of patients receiving combined treatment with either GnRH or orchidectomy and an anti-androgen, such as flutamide, to block the actions of androgens, including adrenal androgens. Such patients have increased progression-free survival time compared to patients treated with GnRH agonist or orchidectomy alone (Crawford et al., N. Engl. J. Med., 1989, 321, 419 and Labrie et al., Cancer Suppl., 1993, 71, 1059).

Although patients initially respond to endocrine therapy, they frequently relapse. It was reported recently that in 30% of recurring tumors of patients treated with endocrine therapy, high-level androgen receptor (AR) amplification was found (Visakorpi, et al., Nature Genetics, 1995, 9, 401). Also, flutamide tends to interact with mutant ARs, and stimulate prostatic cell growth. This suggests that AR amplification may facilitate tumor cell growth in low androgen concentrations. Thus, total androgen blockade as first line therapy may be more effective than conventional androgen deprivation by achieving maximum suppression of androgen concentrations which may also prevent AR amplification. It is presently unclear whether sequential treatment with different agents can prolong the benefits of the initial therapy. This strategy has been found effective in breast cancer treatment. New agents which act by different mechanisms could produce second responses in a portion of relapsed patients. Although the percentage of patients who respond to second-line hormonal therapy may be relatively low, a substantial number of patients may benefit because of the high incidence of prostate cancer. Furthermore, there is the potential for developing more potent agents than current therapies, none of which are completely effective in blocking androgen effects.

The need exists for C17,20 lyase inhibitors that overcome the above-mentioned deficiencies.

### SUMMARY OF THE INVENTION

The invention provides substituted 3-pyridyl oxazole compounds which inhibit the lyase activity of enzymes, e.g., 17α-hydroxylase-C17,20 lyase.

Compounds of the invention have the formula

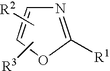

in which

R¹ represents

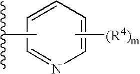

in which R⁴ is selected from C₁₋₆ ally, C₃₋₅ cycloalkyl, CF₃, and

CO₂R⁵, in which R⁵ is H or C₁₋₄ alkyl; and m is 0, 1, or 2;

or

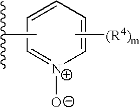

, provided that R³ is other than a pyridyl or an N-oxide-containing group; or

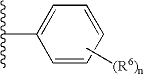

in which R⁶ is selected from C₁₋₄ alkyl, CF₃, OCHF₂, CN,

NO₂, and halogen; and n is 0, 1, or 2.

R² represents H, C alkyl, halogen, or tolyl.

R³ represents

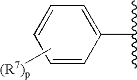

in which

R⁷ is selected from the group consisting of

C₁₋₄ alkyl,

C₁₋₄ alkoxy,

OCHF₂,

halogen,

CF₃,

CN,

phenyl,

NO₂,

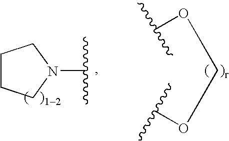

wherein r is 1, 2, or 3, and

N(R⁸)₂ wherein R⁸ is H or C₁₋₄ alkyl, and

p is 0, 1, or 2;

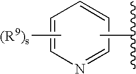

in which R⁹ is C₁₋₄ alkyl or C₃₋₅ cycloalkyl, and

s is 0, 1, or 2; or

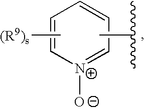

provided that R¹ is other than a pyridyl or an N-oxide-containing group; or

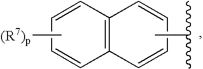

or

C₁₋₄ alkyl.

Further, one of R¹ and R³ is a 3-pyridyl or 3-pyridyl-N-oxide group which is unsubstituted at the 2- and 6-positions. Pharmaceutically acceptable salts of these compounds are also within the scope of the invention.

The invention also provides pharmaceutical compositions for inhibiting lyase activity, comprising a compound of the invention and a pharmaceutically acceptable carrier.

The invention also provides methods for inhibiting lyases, comprising contacting the lyase with a compound of the invention. More particularly, the invention provides a method of inhibiting a 17α-hydroxylase-C17,20 lyase, comprising contacting a 17α-hydroxylase-C17,20 lyase with a compound of the invention.

The invention further provides methods for treating diseases which can benefit from an inhibition of a lyase enzyme. Exemplary diseases are lyase-associated diseases, e.g., diseases resulting from an excess of androgens or estrogens. For example, the invention provides a method for treating cancer in a subject, comprising administering to the subject a pharmaceutically effective amount of a compound of the invention, such that the cancer is treated.

The method of treatment may be applied where the subject is equine, canine, feline, or a primate, in particular, a human.

The cancer may, for example, be prostate or breast cancer. Accordingly, a method for treating prostate cancer in a subject, comprises administering to the subject a therapeutically effective amount of a compound of the invention, such that the prostate cancer in the subject is treated. Similarly, a method for treating breast cancer in a subject comprises administering to the subject a therapeutically effective amount of a compound of the invention, such that the breast cancer in the subject is treated.

### DETAILED DESCRIPTION OF THE INVENTION

The invention is based at least in part on the discovery that substituted 3-pyridyl oxazole compounds inhibit the enzyme 17α-hydroxylase-C17,20 lyase.

In a preferred embodiment, compounds of the invention have the formula

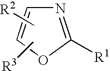

in which

R¹ represents

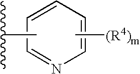

in which R⁴ is selected from C₁₋₆ alkyl, C₃₋₅ cycloalkyl, CF₃;

and m is 0, 1, or 2; or

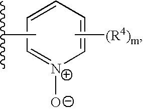

provided that R³ is other than a pyridyl or an N-oxide-containing group; or

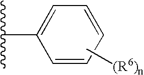

in which R⁶ is selected from C₁₋₄ alkyl, CF₃, OCHF₂, and

halogen; and n is 0, 1, or 2.

R² represents H, C₁₋₆ alkyl, halogen, or tolyl.

R³ represent

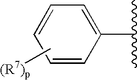

in which

R⁷ is selected from the group consisting of

C₁₋₄ alkyl,

C₁₋₄ alkoxy,

OCHF₂,

halogen,

CF₃,

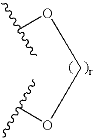

wherein r is 1, 2, or 3, and p is 0, 1, or 2;

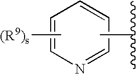

in which R⁹ is C₁₋₄ alkyl or C₃₋₅ cycloalkyl, and s is 0, 1, or 2; or

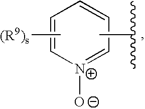

provided that R¹ is other than a pyridyl or an N-oxide-containing group.

Further, one of R¹ and R³ is a 3-pyridyl or 3-pyridyl-N-oxide group which is unsubstituted at the 2- and 6-positions.

In a more preferred embodiment, compounds of the invention have the formula

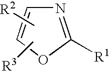

in which

R¹ represents

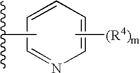

in which R⁴ is selected from C₁₋₆ alkyl and C₃₋₅ cycloalkyl;

and m is 0, 1, or 2; or

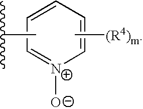

R² represents H.

R³ represents

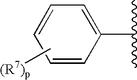

in which

R⁷ is selected from the group consisting of

C₁₋₄ alkyl,

C₁₋₄ alkoxy,

OCHF₂,

halogen, and

p is 0, 1, or 2.

Further, R¹ is a 3-pyridyl or 3-pyridyl-N-oxide group which is unsubstituted at the 2- and 6-positions.

In another more preferred embodiment, compounds of the invention have the formula

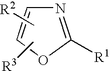

in which

R¹ represents

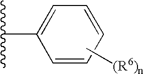

in which R⁶ is selected from CF₃, OCHF₂, and halogen; and

n is 0, 1, or 2.

R² represents H.

R³ represents

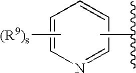

in which R⁹ is C₁₋₄ alkyl or C₃₋₅ cycloalkyl, and s is 0, 1, or 2; or

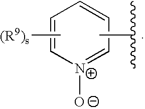

Further, R³ is a 3-pyridyl or 3-pyridyl-N-oxide group which is unsubstituted at the 2- and 6-positions.

Definitions

For convenience, certain terms employed in the specification, examples, and appended claims are collected here.

The term “agonist” of an enzyme refers to a compound that binds to the enzyme and stimulates the action of the naturally occurring enzyme, or a compound which mimics the activity of the naturally occurring enzyme.

The term “antagonist” of an enzyme refers to a compound that binds to the enzyme and inhibits the action of the naturally occurring enzyme.

The term “CYP17 substrate” includes any of the various steroid hormones acted upon by a CYP17 or a CYP17-like P₄₅₀ enzyme. Examples include pregnenolone, progesterone and their 17α-hydroxylated forms. Pregnenolone is converted to DHEA via a CYP17 C17,20-lyase reaction, but is also subject to C17α-hydroxylation via the C17,20-lyase activity. Progesterone is converted to delta 4-androstenedione via a CYP17 C17,20-lyase reaction, but is also subject to C17 alpha-hydroxylation via the C17-hydroxylase activity to form 17-hydroxyl-progesterone, a precursor to hydrocortisone (i.e. cortisol).

The term “CYP17 metabolite” refers to any of the steroid hormones that are synthesized from a cholesterol precursor via a CYP17-mediated reaction, such as a C17-hydroxylase reaction or a C17,20-lyase reaction. Examples of CYP17 metabolites include the androgens, such as testosterone, which are synthesized via a CYP17 C17,20-lyase reaction from CYP17 substrate precursors such as pregnenolone (converted to DHEA by the CYP17 C17,20-lyase activity), and progesterone (converted to delta 4-androstenedione by the CYP17 C17,20-lyase activity). Progestagens such as progesterone are primarily synthesized in the corpus luteum. The androgens are responsible for, among other things, development of male secondary sex characteristics and are primarily synthesized in the testis. Other examples include the estrogens, which are also synthesized from a cholesterol precursor via a CYP17-mediated reaction. The estrogens are responsible for, among other things, the development of female secondary sex characteristics and they also participate in the ovarian cycle and are primarily synthesized in the ovary. Another group of CYP17 metabolites are the glucocorticoids, such as hydrocortisone (i.e. cortisol), which is synthesized from progesterone via a CYP17-mediated reaction. The glucocorticoids, among other functions, promote gluconeogenesis and the formation of glycogen and also enhance the degradation of fat. The glucocorticoids are primarily synthesized in the adrenal cortex.

The term “CYP17 metabolite” is further meant to include other steroid hormones which, although not necessarily synthesized by a CYP17-mediated reaction, may nonetheless be understood by the skilled artisan to be readily affected by an alteration in a CYP17-mediated activity. For example, the mineralocorticoids, such as aldosterone, are derived from cholesterol via a progesterone intermediate. Since progesterone is also converted to the glucocorticoids and sex steroids via CYP17-mediated reactions, an alteration of a CYP17 activity can alter the amount of progesterone available for conversion to aldosterone. For example, inhibition of CYP17 activity can increase the amount of progesterone available for conversion into aldosterone. Therefore, inhibition of CYP17 can lead to an increase in the level of aldosterone. The mineralocorticoids function, among other things, to increase reabsorption of sodium ions, chloride ions, and bicarbonate ions by the kidney, which leads to an increase in blood volume and blood pressure. The mineralocorticoids are primarily synthesized in the adrenal cortex.

The term “CYP17 metabolite-associated disease or disorder” refers to a disease or disorder which may be treated by alteration of the level of one or more CYP17 metabolites. Examples include a hormone dependent cancer, such as an androgen-dependent prostate cancer, which may be treated by inhibiting CYP17-mediated androgen synthesis, and an estrogen-dependent breast cancer or ovarian cancer, which may be treated by inhibiting CYP17-mediated estrogen synthesis. Other examples of “CYP17 metabolite-associated diseases or disorders” are Cushing's disease, hypertension, prostatic hyperplasia, and glucocorticoid deficiency. Patients with Cushing's syndrome are relatively insensitive to glucocorticoid feedback and exhibit an oversecretion of cortisol devoid of a circadian cycle (see e.g. Newell-Price & Grossman (2001) Ann. Endocrinol. 62: 173-9). Another CYP17 metabolite-associated disease or disorder is hypertension. Mineralocorticoid excess causes hypertension by facilitating the sodium retention at renal tubules.

“Disease associated with an abnormal activity or level of a lyase” refers to diseases in which an abnormal activity or protein level of a lyase is present in certain cells, and in which the abnormal activity or protein level of the lyase is at least partly responsible for the disease.

A “disease associated with a lyase” refers to a disease that can be treated with a lyase inhibitor, such as the compounds disclosed herein.

A “lyase” refers to an enzyme having a lyase activity.

“Lyase activity” refers to the activity of an enzyme to catalyze the cleavage of the bond C17-C20 in 17α-hydroxy-pregnenolone and 17α-hydroxy-progesterone to form dehydroepiandrosterone (DHEA) and delta4-androstenedione, respectively. Lyase activity also refers to the cleavage of a similar bond in related compounds.

A “lyase inhibitor” is a compound which inhibits at least part of the activity of a lyase in a cell. The inhibition can be at least about 20%, preferably at least about 40%, even more preferably at least about 50%, 70%, 80%, 90%, 95%, and most preferably at least about 98% of the activity of the lyase.

A “patient” or “subject” to be treated by the subject method can mean either a human or non-human animal.

“Treating” a disease refers to preventing, curing or improving at least one symptom of a disease.

The following definitions pertain to the chemical structure of compounds:

The term “heteroatom” as used herein means an atom of nitrogen, oxygen, or sulfur.

The term “alkyl” refers to the radicals of saturated aliphatic groups, including straight-chain alkyl groups and branched-chain alkyl groups.

The term “cycloalkyl” (alicyclic) refers to radicals of cycloalkyl compounds, examples being cyclopropyl, cyclobutyl, cyclopentyl, cyclohexyl, etc.

The term “aralkyl”, as used herein, refers to an alkyl group substituted with an aryl group (e.g., an aromatic or heteroaromatic group).

The terms “alkenyl” and “alkynyl” refer to unsaturated aliphatic groups that contain at least one double or triple bond respectively.

Unless the number of carbons is otherwise specified, “lower alkyl” as used herein means an alkyl group but having from one to six carbons, preferably from one to four carbon atoms in its backbone structure. Likewise, “lower alkenyl” and “lower alkynyl” have similar chain lengths. Preferred alkyl groups are lower alkyls.

The term “aryl” as used herein means an aromatic group of 6 to 14 carbon atoms in the ring(s), for example, phenyl and naphthyl. As indicated, the term “aryl” includes polycyclic ring systems having two or more rings in which two or more carbons are common to two adjoining rings (the rings are “fused rings”) wherein at least one of the rings is aromatic.

The term “heteroaryl” as used herein means an aromatic group which contains at least one heteroatom in at least one ring. Typical examples include 5-, 6- and 7-membered single-ring aromatic groups that may include from one to four heteroatoms. Examples include pyrrole, furan, thiophene, imidazole, oxazole, thiazole, triazole, tetrazole, pyrazole, pyridine, pyrazine, pyridazine and pyrimidine, and the like. These aryl groups may also be referred to as “aryl heterocycles” or “heteroaromatics.”

The terms ortho, meta and para apply to 1,2-, 1,3- and 1,4-disubstituted benzenes, respectively. For example, the names 1,2-dimethylbenzene and ortho-dimethylbenzene are synonymous.

The terms “alkoxyl” or “alkoxy” as used herein refer to moiety in which an alkyl group is bonded to an oxygen atom, which is in turn bonded to the rest of the molecule. Examples are methoxy, ethoxy, propyloxy, tert-butoxy, etc.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms triflyl, tosyl, mesyl, and nonaflyl are art-recognized and refer to trifluoromethanesulfonyl,p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf Nf, Ts, Ms represent methyl, ethyl, phenyl, trifluoromethanesulfonyl, nonafluorobutanesulfonyl,p-toluenesulfonyl and methanesulfonyl, respectively. A more comprehensive list of the abbreviations utilized by organic chemists of ordinary skill in the art appears in the first issue of each volume of the Journal of Organic Chemistry; see for example, 2002, 67(1), 24A. The abbreviations contained in said list are hereby incorporated by reference.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 3^{rd} ed.; Wiley: New York, 1999).

### Abbreviations and Acronyms

When the following abbreviations are used throughout the disclosure, they have the following meaning:

AcOH acetic acid

Ar argon

BSA bovine serum albumin

n-BuLi butyllithium

CDCl₃ chloroform-d

CD₃OD methanol-d₄

CHCl₃ chloroform

CH₂Cl₂ methylene chloride

CH₃CN acetonitrile

CuI copper iodide

Cs₂CO₃ cesium carbonate

CPM counts per minute

DMW dimethylformamide

DMSO dimethylsulfoxide

EPA Environmental Protection Agency (as in EPA vial)

ESI Electrospray ionization

Et₃N triethylamine

EtOAc ethyl acetate

Et₂O diethyl ether

EtOH ethanol

GCEI gas chromatography—electron impact mass spectrometry

GCMS gas chromatography/mass spectrometry

H₂ hydrogen gas

HCl hydrochloric acid

¹H NMR proton nuclear magnetic resonance

HEPES 4-(2-Hydroxyethyl)piperazine-1-ethanesulfonic acid

HPLC high performance liquid chromatography

KOH potassium hydroxide

LCMS liquid chromatography/mass spectroscopy

MeCN acetonitrile

MeOH methanol

Min minute

mol mole

NaHCO₃ sodium bicarbonate

NaHMDS sodium bis(triiethylsilyl)amide

NaN₃ sodium azide

Na₂SO₄ sodium sulfate

NH₄Cl ammonium chloride

NH₄OH ammonium hydroxide

nm nanomolar

Pd/C palladium on carbon

POCl₃ Phosphorous oxychloride

Rf TLC retention time

rt room temperature

Rₜ retention time

SPA Scintillation Proximity Assay

THF tetrahydrofuran

TFA trifluoroacetic acid

TMS tetramethylsilane

TLC thin layer chromatography

t_{R} retention time

Compounds of the Invention

The present invention is directed to compounds which inhibit 17α-hydroxylase-C17,20-lyase.

Certain compounds of the present invention may exist in particular geometric or stereoisomeric forms. The present invention contemplates all such compounds, including cis- and trans-isomers, R— and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an allyl group. All such isomers, as well as mixtures thereof, are intended to be included in this invention.

If, for instance, a particular enantiomer of a compound of the present invention is desired, it may be prepared by asymmetric synthesis, or by derivatixaton with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively nontoxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

Pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pannitic, maleic, hydroxymaleic, phenylacetic, glutamic, benzoic, salicyclic, sulfanilic; 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. These salts can be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Contemplated equivalents of the compounds described above include compounds which otherwise correspond thereto, and which have the same general properties thereof (e.g., functioning as 17α-hydroxylase-C17,20-lyase inhibitors), wherein one or more simple variations of substituents are made which do not adversely affect the efficacy of the compound in binding to 17α-hydroxylase-C17,20-lyase receptors.

In general, the compounds of the present invention may be prepared by the methods illustrated in the general reaction schemes given below, by the Examples, or by modifications thereof, using readily available starting materials, reagents and conventional synthesis procedures. In these reactions, it is also possible to make use of variants which are in themselves known, but are not mentioned here.

Diseases That Can be Treated With the Compounds of the Invention

The present invention provides a method of inhibiting a lyase, e.g., 17α-hydroxylase-C17,20 lyase, comprising contacting a lyase with a compound of the invention. The activity can be inhibited by at least 20%, preferably at least about 50%, more preferably at least about 60%, 70%, 80%, 90%, 95%, and most preferably at least about 98%. In one embodiment, the invention provides a method for inhibiting a lyase in vitro. In a preferred ernbodiment, the lyase is in vivo or ex vivo. For example, the invention provides methods for inhibiting a lyase in a cell, comprising contacting the cell with a compound of the invention, such that the activity of the lyase is inhibited. The cell may further be contacted with a composition stimulating the uptake of the compound into the cell, e.g., liposomes. In one embodiment, the invention provides a method for inhibiting a lyase in a cell of a subject, comprising administering to the subject a therapeutically effective amount of a compound of the present invention, or a formulation comprising a compound of the present invention, such that the lyase is inhibited in a cell of the subject. The subject can be one having a disease associated with a lyase, e.g., cancer. Types of cancer that can be treated according to the invention preferably include prostate cancer and breast cancer. Other diseases that can be treated include diseases in which it is desired to prevent or inhibit the formation of a hormone selected from the group consisting of the androgens testosterone and dihydrotestosterone (DHT) and the estrogens 17β-estradiol and estrone. Generally, any disease that can be treated by inhibiting the activity of a lyase, e.g., 17α-hydroxylase-C17,20-lyase, can be treated with the compounds of the invention.

In general, the invention provides methods and compositions for the treatment of CYP17 metabolite-associated diseases and disorders. Examples include particularly sex steroid hormone dependent cancers, such as androgen-dependent prostate cancer, which may be treated by inhibiting CYP17-mediated androgen synthesis, and estrogen-dependent breast cancer or ovarian cancer, which may be treated by inhibiting CYP17-mediated estrogen synthesis.

For example, adenocarcinoma of the prostate is a common disease that causes significant morbidity and mortality in the adult male population (see Han and Nelson (2000) Expert Opin. Pharmacother. 1: 443-9). Hormonal therapy for prostate cancer is considered when a patient fails with initial curative therapy, such as radical prostatectomy or definitive radiation therapy, or if he is found with an advanced disease. Hormonal agents have been developed to exploit the fact that prostate cancer growth is dependent on androgen. Non-steroidal anti-androgens (NSAAs) block androgen at the cellular level. Castration is another, albeit drastic means of decreasing androgens levels in order to treat or prevent prostate cancer. The methods and compositions of the invention are useful in inhibiting the C17,20-lyase activity of CYP17 and thereby decreasing levels of androgen production and the associated growth of androgen-dependent cancers such as prostate cancer.

In another example, breast cancer, particularly breast cancer in postmenopausal women, can be treated by administration of a C17,20-lyase inhibitor of the invention because adrenal and ovarian androgens are the main precursors of the estrogens which stimulate the growth of hormone dependent breast cancer. In addition, breast cancer can be treated with inhibitors of aromatase that prevent interconversion of estrogens and adrenal and ovarian androgens (see Harris et al. (1983) Eur. J. Cancer Clin. Oncol. 19: 11). Patients failing to respond to aromatase inhibitors show elevated levels of androgens in response to aromatase inhibitor treatment (see Harris et al. (1988) Br. J. Cancer 58: 493-6). Accordingly sequential blockade to inhibit androgen production as well as inhibit aromatase may produce greater estrogen suppression and enhanced therapeutic effects in treating breast and other estrogen hormone-dependent forms of cancer. Therefore the inhibitors of the invention may be used alone or in combination with other drugs to treat or prevent hormone-dependent cancers such as breast and prostate cancer.

Furthermore, susceptibility to prostate cancer and breast cancer has been associated with particular polymorphic alleles of the CYP17 gene (see e.g. McKean-Cowdin (2001) Cancer Res. 61: 848-9; Haiman et al. (2001) Cancer Epidmeiol. Biomarkers 10: 743-8; Huang et al. (2001) Cancer Res. 59: 4870-5). Accordingly, the compositions of the invention are particularly suited to treating or preventing hormone-dependent cancers in individuals genetically predisposed to such cancers, particularly those predisposed due to an alteration in the CYP17 gene.

Another group of CYP17 metabolite-associated diseases or disorders amenable to treatment with the compositions and methods of the invention include those associated with mineralocorticoid excess such as hypertension caused by sodium retention at renal tubules. Such a mechanism operates in hypertension such as primary hyperaldosteronism and some forms of congenital adrenal hyperplasia Recently, deficient cortisol metabolism in the aldosterone target organ has been recognized as a novel form of hypertension known as apparent mineralocorticoid excess. Disorders associated with mineralocorticoid synthesis include abnormalities of mineralocorticoid synthesis and/or metabolism which profoundly affect the regulation of electrolyte and water balance and of blood pressure (see e.g. Connell et al. (2001) Baillieres Best Pract. Res. Clin. Endocrinol. Metab. 15:43-60). Characteristic changes in extracellular potassium, sodium and hydrogen ion concentrations are usually diagnostic of such disorders. Serious deficiency may be acquired, for example, in Addison's disease, or inherited. In most of the inherited syndromes, the precise molecular changes in specific steroidogenic enzymes have been identified. Mineralocorticoid excess may be caused by aldosterone or 11-deoxycorticosterone by inadequate conversion of cortisol to cortisone by 11β-hydroxysteroid dehydrogenase type 2 in target tissues, by glucocorticoid receptor deficiency or by constitutive activation of renal sodium channels. Changes in electrolyte balance and renin as well as the abnormal pattern of corticosteroid metabolism are usually diagnostic. Where these abnormalities are inherited (e.g. 11beta- or 17alpha-hydroxylase deficiencies, glucocorticoid remediable hyperaldosteronism (GRA), receptor defects, Liddle's syndrome), the molecular basis is again usually known and, in some cases, may provide the simplest diagnostic tests. Primary aldosteronism, although readily identifiable, presents problems of differential diagnosis, important because optimal treatment is different for each variant. Finally, a significant proportion of patients with essential hypertension show characteristics of mild mineralocorticoid excess, for example low renin levels. As described above, a decrease in CYP17 activity can result in an alteration in mineralorticoid (e.g. aldosterone) biosynthesis. Accordingly, the “CYP17 metabolite-associated diseases or disorders” of the invention would include those associated with altered levels of aldosterone production (e.g. hypertension, primary adrenal hyperplasia).

Still other examples of CYP17 metabolite-associated diseases or disorders” are Cushing's disease, prostatic hyperplasia, glucocorticoid deficiency, and endometrial cancer.

The subject that can be treated according to the invention can be a mammal, e.g., a primate, equine, canine, bovine, ovine, porcine, or feline. In preferred embodiments of this method, the mammal is a human. In other embodiments, the invention provides methods for inhibiting the lyase activity of enzymes that are present in organisms other than mammals, e.g., yeast and fungus, e.g., mildew. Certain compounds of the invention may function as antifungal compounds.

Methods of Administering the Compounds of the Invention

The therapeutic methods of the invention generally comprise administering to a subject in need thereof, a pharmaceutically effective amount of a compound of the invention, or a salt, prodrug or composition thereof. The compounds of the invention can be administered in an amount effective to inhibit the activity of a 17α-hydroxylase-C17,20-lyase. The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenteraly, including the intravenous, intramuscular, intraperitoneal, subcutaneous, rectal and topical routes of administration.

Toxicity and therapeutic efficacy of the compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such reagents to the site of affected tissue in order to minimize potential damage to normal cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of activity) as determined in cell culture. Such information can be used to more accurately determine useful doses in humans. The compounds of the invention have an IC₅₀ less than 10 μM as determined by the biochemical or cellular assay described herein. Some compounds of the invention are effective at concentrations of 10 nM, 100 nM, or 1 μM. Based on these numbers, it is possible to derive an appropriate dosage for administration to subjects.

Formation of prodrugs is well known in the art in order to enhance the properties of the parent compound. Such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995)). Commonly used prodrugs of the disclosed compounds can be designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996)).

The pharmaceutical compositions can be prepared so that they may be administered orally, dermally, parenterally, nasally, ophthalmically, otically, sublingually, rectally or vaginally. Dermal administration includes topical application or transdermal administration. Parenteral administration includes intravenous, intraarticular, intramuscular, intraperitoneal, and subcutaneous injections, as well as use of infusion techniques. One or more compounds of the invention may be present in association with one or more non-toxic pharmaceutically acceptable ingredients and optionally, other active anti-proliferative agents, to form the pharmaceutical composition. These compositions can be prepared by applying known techniques in the art such as those taught in Remington's Pharmaceutical Sciences (Fourteenth Edition), Managing Editor, John E. Hoover, Mack Publishing Co., (1970) or Pharmaceutical Dosage Form and Drug Delivery Systems (Sixth Edition), edited by Ansel et al., publ. by Williams & Wilkins, (1995).

As indicated above, pharmaceutical compositions containing a compound of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically acceptable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia; and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally occurring phosphatide, for example lecithin; or condensation products of an alkylene oxide with fatty acids, for example polyoxyethylene stearate; or condensation products of ethylene oxide with long chain aliphatic alcohols, for example heptadecaethylene-oxycetanol; or condensation products of ethylene oxide with partial esters derived from fatty acids and a hexitol such as polyoxyethylene sorbitol monooleate; or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the compound of the invention in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present These compositions may be preserved by the addition of an anti-oxidant such as ascorbic acid.

Pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solutions. Among the acceptable vehicles and solvents that may be employed are water, Ringer's solution and isotonic sodium chloride solution.

Sterile injectable preparation may also be a sterile injectable oil-in-water microemulsion where the compound of the invention is dissolved in the oily phase. For example, the active ingredient may be first dissolved in a mixture of soybean oil and lecithin. The oil solution is then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the active compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a nontoxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of a suppository for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of the invention can be employed. For purposes of this application, topical application shall include mouth washes and gargles.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will preferably be continuous rather than intermittent throughout the dosage regimen.

The compounds of the invention may also be co-administered with other well known therapeutic agents that are selected for their particular usefulness against the condition that is being treated. The compounds may be administered simultaneously or sequentially. For example, the active compounds may be useful in combination with known anti-cancer and cytotoxic agents. Similarly, the active compounds may be useful in combination with agents that are effective in the treatment and prevention of osteoporosis, inflammation, neurofibromatosis, restinosis, and viral infections. The active compounds may also be useful in combination with inhibitors of other components of signaling pathways of cell surface growth factor receptors.

Drugs that can be co-administered to a subject being treated with a compound of the invention include antineoplastic agents selected from vinca alkaloids, epipodophyllotoxins, anthracycline antibiotics, actinomycin D, plicamycin, puromycin, gramicidin D, taxol, colchicine, cytochalasin B, emetine, maytansine, or amsacrine. Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), 1996 edition (Medical Economics Company, Montvale, N.J. 07645-1742, USA).

Radiation therapy, including x-rays or gamma rays which are delivered from either an externally applied beam or by implantation of tiny radioactive sources, may also be used in combination with a compound of the invention to treat a disease, e.g., cancer.

When a composition according to this invention is administered into a human subject, the daily dosage will normally be determined by the prescribing physician with the dosage generally varying according to the age, weight, and response of the individual patient, as well as the severity of the patient's symptoms.

Kits of the Invention

In one embodiment, a compound of the invention, materials and/or reagents required for administering the compounds of the invention may be assembled together in a kit. When the components of the kit are provided in one or more liquid solutions, the liquid solution preferably is an aqueous solution, with a sterile aqueous solution being particularly preferred.

The kit may further comprise one or more other drugs, e.g., a chemo- or radiotherapeutic agent. These normally will be a separate formulation, but may be formulated into a single pharmaceutically acceptable composition. The container means may itself be geared for administration, such as an inhalant, syringe, pipette, eye dropper, or other such like apparatus, from which the formulation may be applied to an infected area of the body, such as the lungs, or injected into an animal, or even applied to and mixed with the other components of the kit.

The compositions of these kits also may be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the agent. Kits may also comprise a compound of the invention, labeled for detecting lyases.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with a separate instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of reactions or amounts of compounds or polypeptides.

The present invention is further illustrated by the following examples which should not be construed as limiting in any way. The contents of all cited references (including literature references, issued patents, published patent applications as cited throughout this application) are hereby expressly incorporated by reference.

Determination of the Activity of the Compounds of the Invention

C17,20 Lyase inhibitory activity of compounds can be determined using, e.g., the biochemical or the cellular assays set forth in the Examples. A person of skill in the art will recognize that variants of these assays can also be used.

The compounds of the invention can also be tested in animal models, e.g., animal models of prostate or breast cancer.

Each of the compounds of the invention was subjected to a biochemical assay and a cellular assay for determining its C17,20 lyase inhibitory activity.

### Human and Murine C17,20-lyase Biochemical Assays

Recombinant human C17,20-lyase (hLyase) was expressed in baculovirus-infected Sf9 cells and hLyase enriched microsomes were prepared from cultures as described (Barnes H. J.; Jenkins, C. M.; Waterman, M. R. Archives of Biochemistry and Biophysics 1994, 315(2), 489-494). Recombinant murine C17,20-lyase (mLyase) was prepared in a similar manner. hLyase and naLyase preparations were titrated using assay conditions to determine protein concentrations to be used for assays. Both mLyase and hlyase assays were run in an identical manner except that cytochrome b5 was omitted in the murine assay.

Test compound solutions (20 mM in DMSO) were diluted 1:4 with DMSO and put into the top well of a 96-well mother plate. These solutions were then diluted serially in six steps (1:4 each step) with DMSO to obtain 800 μM to 51.2 nM concentrations on a mother plate (columns 3-12) for subsequent use in the assay. These compound solutions were further diluted twenty- fold in water to obtain a daughter plate containing compound concentrations ranging from 40 μM to 2.56 nM in 5% DMSO. The first 2 columns (of wells) on each 96-well mother plate were used for the DHEA (dehydroepiandrosterone) standard curve. DHEA standards were serially diluted (in half-logs) in DMSO to obtain 400 μM to 120 nM standards, then diluted (1:19) in water to obtain 20 μM to 6 nM solutions in 5% DMSO on the daughter plate. These 5% DMSO solutions (5 μL each) from the daughter plate were transferred to the SPA assay plate prior to adding the reaction mixture.

To prepare the reaction mixture, clear-bottomed opaque 96-well assay plates were loaded with 50 μL of assay buffer (50 mM Na₃PO₄, pH 7.5), 5 μL of the diluted compounds (or standards), and 30 μL of substrate solutions (7 mM NADPH, 3.35 μM 17-OH-pregnenolone, 3.35 μg/mL human cytochrome b₅ in 50 mM Na₃PO₄). Reactions were initiated with the addition of hLyase or mLyase in assay buffer (10 μL). Enzymatic reactions were incubated at room temperature for 2 hours with gentle agitation. Reactions were terminated with the addition of 5 μL of 1 mM (50 μM final concentration) YM116, a potent C17,20-lyase inhibitor.

The concentration of DHEA generated by hLyase (or mLyase) was determined by radioimmunoassay (RIA). RIA utilized a ³H-DHEA (0.08 μCi) tracer in 50 μL of scintillation proximity assay (SPA) buffer (100 mM Tris-HCl, pH 7.5, 50 mM NaCl, 0.5% BSA, 0.2% Tween 20) which was added to each well. DHEA antiserum from rabbit (50 μL) with anti-rabbit SPA beads in SPA buffer was added to all wells. Mixtures were allowed to equilibrate with gentle agitation for 1 hour followed by overnight equilibration with no agitation. ³H-DHEA bound to the SPA beads was determined by scintillation counting with a Wallac microbeta counter. The concentration of DHEA generated was calculated from raw data (CPM) and the standard curve. The concentration of DHEA formed in the presence of test compounds was expressed as a percent inhibition compared to the DHEA concentration in the absence of test compounds: [1-(nM DHEA formed in the presence of test compound/nM DHEA formed in the absence of test compounds)]×100. Determination of IC₅₀ for each compound was performed using the Analyze 5 program.

### Human C17,20-lyase Cellular Assay

Human HEK 293-lyase stable transfectant cells were seeded in a 96-well plate at 10,000 cells/well/100 μL in DMEM plus 10% FBS (supplemented with 1% glutamine, 0.8 mg/mL G418) and allowed to attach overnight. On the following day, the media was removed from the cell plate and replaced with 100 μL RPMI without phenol red. Test compounds (columns 3-12), DMSO vehicle (column 2), or DHEA standards (column 1) of 5 μL each were added to the cell plate and incubated for 10 min. at room temperature. The final concentrations of DHEA standards were 750, 250, 83.3, 27.7, 9.2, 3, 1, and 0.3 nM. The reaction was initiated with 10 μL of 5 μM 17-OH-pregnenolone being added to all the wells of the cell plate, then incubated for 1 hour at 37° C. Following the incubation, 90 μL of media (containing DHEA product) was removed from the cell plate and transferred to the SPA assay plate. The subsequent SPA procedure for the detection of DHEA product was performed in the same manner as described for the enzyme assay (see above). The mother plate of test compounds was also prepared in the same manner as the enzyme assay. However, the highest concentration of compounds on the daughter plate was 200 μM rather than 40 μM, such that the highest dose of compound tested was 10 μM in final concentration (cellular assay) rather than 2 μM (biochemical assay).

Reagents (including catalog #) for the SPA assay were obtained from the following sources: ³H-DHEA: NEN (NET814), Anti-DHEA: Endocrine Sciences (D7-421), Anti-Rabbit SPA Beads: Amersham (RPNQ 0016), 17-OH-pregnenolone: Steraloids (Q4710), NADPH: Sigma (N1630), Cytochrome b5: Panvera (P2252), DHEA (500 μM stock in 100% EtOH), BSA: Sigma (A9647).

A test compound was considered to be active if the IC₅₀ in the human C17,20 biochemical assay or in the human C17,20 cellular assay was less than 10 μM. All the compounds tested have IC₅₀ in the human C17,20 biochemical assay or the human C17,20 cellular assay of less than 10 μM.

The present invention is further illustrated by the following examples, which should not be construed as limiting in any way. The contents of all cited references (including literature references, issued patents, published patent applications as cited throughout this application) are hereby expressly incorporated by reference.

Preparation of the Compounds of the Invention

General. All reagents are commercially available unless otherwise specified. Reagents were used as received unless otherwise specified. Proton NMR data is reported downfield from TMS. Mass spectral data (LC/MS) were obtained using a Hewlett-Packard 1100 HPLC equipped with a quaternary pump, a variable wavelength detector set at 254 nm, a YMC pro C-18 column (2×23 mm, 120 A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonirile with 0.02% TFA. Gradient elution from 10% B to 95% B over 3.5 minutes at a flowrate of 1.0 mL/min was used with an initial hold of 0.5 minutes and a final hold at 95% B of 0.5 minutes. Total run time was 6.5 minutes. Purification by HPLC was performed using a Gilson HPLC system (UV/VIS-155 detector, 215 liquid handler, 306 pumps, 819 injection valve and an 811C mixer; the column was a YMC Pro C18 (20×150 mm, 5 um, 120 A); the eluents were A: water with 0.1% TFA, and B: acetonitrile with 0.1% TFA; gradient elution; flow rate 20 mL per minute), unless otherwise indicated. Elemental analyses were obtained at Robertson Microlit Laboratories, Madison N.J. Melting points are uncorrected.

### General Method for the Synthesis of 4-Substituted Nicotinic Acid Derivatives

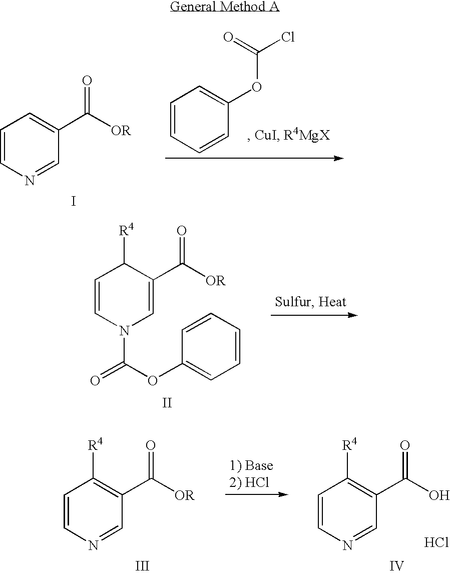

General Method A: The reaction conditions for the synthesis of 4-substituted nicotinic acids IV are similar to those described by Comins, D. L., Smith, R., Stroud, E., Heterocycles, Vol. 22, No. 2, 1984, 339. A 3-nicotinate ester I is converted to the 1, 3, 4 trisubstituted dihydronicotinate II by reacting with a phenylchloroformate, CuI, and an alkyl magnesium halide. Heating II in the presence of sulfur affords the re-aromatized pyridine of the formula III. Hydrolysis, followed by HCl salt formation, results in the formation of the 4-substituted nicotinic acid derivative of the formula IV.

Pyridine-3,4-dicarboxylic acid 4-methyl ester is obtained by conditions described in Moore, B., McLaughlin, R.; Urban, F., Young, G. Organic Preparations and Procedures, International, Vol. 28, No. 3, 1996, 360.

Method A. The syntheses of compounds of the formula IV are exemplified by the synthesis of 4-ethyl nicotinic acid hydrochloride.

Preparation of 4-ethyl nicotinic acid hydrochloride.

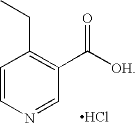

### Step 1. Preparation of methyl-4-ethyl nicotinate, B

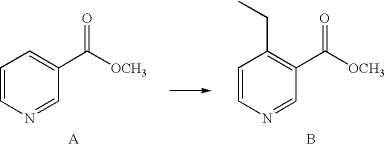

A solution of methyl nicotinate, A (20 g, 0.146 mol), dimethyl sulfide (70.5 mL, 0.96 mol) and copper (I) iodide (1.39 g, 0.0073 mol) in anhydrous THF (200 mL) was stirred at room temperature under an argon atmosphere. Phenyl chloroformate (19.5 mL, 0.155 mol) was then added. After 30 minutes, the mixture was cooled below −21° C. and ethyl magnesium bromide (3.0 M in diethyl ether, 49 mL, 0.146 mol) was added over 20 minutes, keeping the reaction temperature below −25° C. The mixture was stirred and allowed to warm slowly; after 2 hours it had warmed to 8.8° C. 20% aqueous ammonium chloride solution (140 mL) was added; after stirring 20 minutes, the mixture was poured into a separatory funnel. Aqueous layer was washed with ethyl acetate (30 ml) and the combined organic layer then washed with 20% aqueous ammonium chloride solution (70 mL), dried (Na₂SO₄), filtered and then concentrated in vacuo. The residue was purified by silica gel chromatography using a hexane-EtOAc gradient to afford 35.41 g (84%) of the intermediate dihydropyridine.

To a solution of the intermediate dihydropyridine (35.41 g, 0.123 mol) in decalin (142 mL) was added sulfur (3.94 g, 0.123 mol) and the suspension was slowly heated to reflux under an argon sweep. After refluxing 1 h, the mixture was allowed to cool to room temperature, then filtered through a pad of silica gel. After eluting the decalin with hexane, elution with a hexane-ethyl acetate gradient afforded 9.02 g (45%) of methyl-4-ethyl-nicotinate, B as a yellow oil: Rf=0.27 (30% EtOAc/Hexanes); ¹H NMR (CDCl₃) δ 9.0 (s, 1H), 8.6 (d, 1H), 7.2 (d, 1H), 3.9 (s, 3H), 3.0 (q, 2H), 1.25 (t, 3H); LC/MS (RT 0.87) 166 (MH³⁰).

### Step 2. Preparation of 4-ethyl nicotinic acid hydrochloride, C

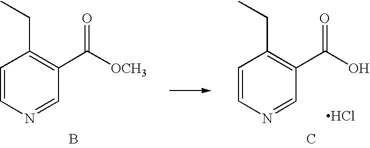

A mixture of methyl-4ethyl nicotinate, B, (6.8 g, 0.041 mol) in THF (62 ml) and 1N NaOH (61.5 ml, 0.0615 mol) was stirred at room temperature for 2 hours. The reaction was judged complete by TLC (30% ethyl acetate/hexane). The reaction mixture was then concentrated to remove the organic solvent. The aqueous solution was washed with dichloromethane (1×75 ml). The aqueous solution was then poured over ion exchange resin (Amberlite IRA-400 (Cl)) and the column was washed with water (2×500 ml). The product was eluted by using a gradient of 1-6N hydrochloric acid. Concentration of the fractions, followed by drying under vacuum at 35-40° C. gave 3.38 g (55%) of 4-ethyl nicotinic acid, hydrochloride salt, C. ¹H NMR (DMSO-d6) δ 9.1 (s, 1H), 8.83 (d, 1H), 7.83 (d, 1H), 3.1 (q, 2H), 1.2 (t, 3H); LC/MS (Rₜ 0.75) 152 (MH⁺).

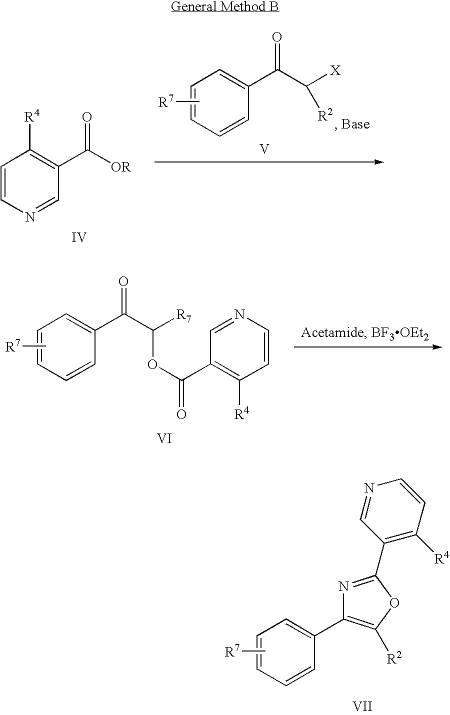

General Method B: The oxazoles of formula VII are prepared by the reaction sequence described in General Method B. 4-substituted nicotinic acids IV are available commercially or are readily prepared in accordance with the scheme for General Method

A. The nicotinic acid IV is converted to the ester of type VI by coupling with a alpha-halo ketone V in the presence of base. Heating the ester VI with acetamide and BF₃.OEt₂ in an appropriate solvent, such as xylene, affords the oxazole of type VII.

Method B. The syntheses of compounds of the formula VII are exemplified by the synthesis of 3-[4-(3-Bromo-phenyl)-oxazole-2-yl]-4-methyl-pyridine, example 1.

### EXAMPLE 1

### Preparation of 3-[4-(3-Bromo-phenyl)-oxazole-2-yl]-4-methyl-pyridine

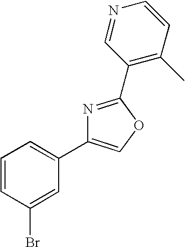

### Step 1. Preparation of 2-(3-bromophenyl)-2-oxoethyl 4-methylpyridine-3-carboxylate

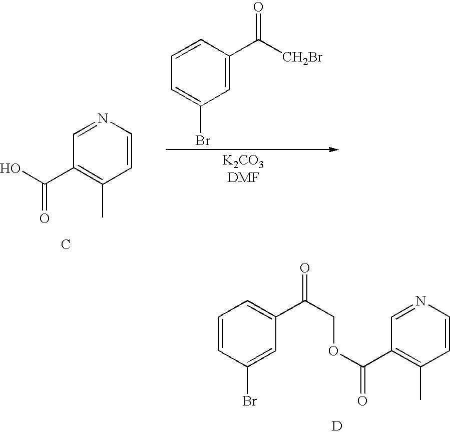

3-Bromophenacyl bromide (5.0 g; 18 mmol) was added to a stirred suspension 4-methyl nicotinic acid hydrochloride, C (3.1 g, 18 mmol) and potassium carbonate (5.0 g, 36 mmol) in N,N-dimethylforamide (30 mL). The resulting mixture was heated at 55° C. for 1 hour. After cooling to ambient temperature, water was added. The aqueous layer was extracted with Ethyl Acetate (3×). The combined organic layers were dried (MgSO₄), and concentrated. The residue was purified by a silica gel column chromatography to afford the pure product 2-(3-bromophenyl)-2-oxoethyl 4-methylpyridine-3-carboxylate, D (4.1 g, 68%). NMR is consistent with the product 2-(3-bromophenyl)-2-oxoethyl 4-methylpyridine-3-carboxylate, D.

### Step 2. Preparation of 3-[4-(3-Bromo-phenyl)-oxazole-2-yl]4methyl-pyridine, example 1

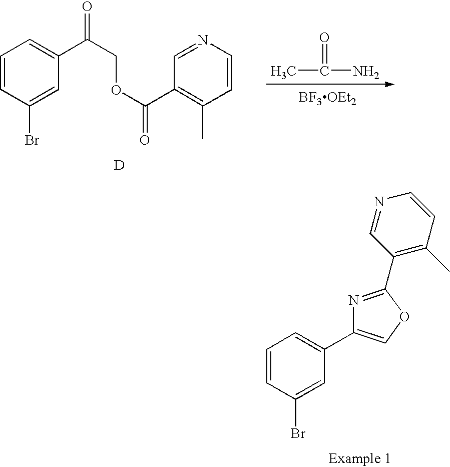

To a mixture of 2-(3-bromophenyl)-2-oxoethyl 4-methylpyridine-3-carboxylate, D (4.1 g, 12.3 mmol) and acetamide (7.3 g, 123 mmol) in p-xylene (35 mL), BF₃.OEt₂ (6.2 mL, 50 mmol) was added. The mixture was refluxed for 18 hours. After cooling to ambient temperature, NaHCO₃ aqueous solution was added. The aqueous layer was extracted with EtOAc (3×), and combined organic layers were dried and concentrated. The residue was purified by a silica gel column chromatography to afford the pure product 3-[4-(3-bromo-phenyl)-oxazol-2-yl]-4-methyl-pyridine, 1 (1.3 g, 28%) as a solid. ¹H-NMR (CD₃OD, 400 MHz) δ 2.8 (s, 3H), 7.35 (t, 1H), 7.5 (m, 2H), 7.82 (d, 1H), 8.08 (s, 1H), 8.50 (m, 2H), 9.10 (s, 1H). LC/MS [M+1], 315.3.

TABLE 1
The following exemplary compounds 2-36 of the formula VII, shown in the
scheme General Method B, were prepared according to method B (example 1) using
appropriately modified reagents.

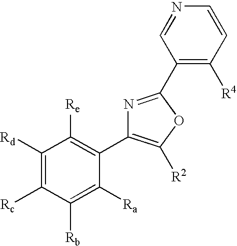

| Entry | R⁴ | Rₐ | R_{b} | R_{c} | R_{d} | Rₑ | R² | Characterization* |
|---|---|---|---|---|---|---|---|---|
| 2 | CH₃ | H | H | OCHF₂ | H | H | H | (M + H)⁺ 303.2 |
| | | | | | | | | R_{f} = 0.49 (55% |
| | | | | | | | | EtOAc/Hexane) |
| 3 | CH₃ | H | H | CN | H | H | H | (M + H)⁺ 262.3 |
| | | | | | | | | R_{f} = 0.32 (1/1 |
| | | | | | | | | EtOAc/Hexane) |
| 4 | CH₃ | H | Cl | Cl | H | H | H | (M + H)⁺ 305.3 |
| | | | | | | | | R_{f} = 0.45 (1/1 |
| | | | | | | | | EtOAc/Hexane) |
| 5 | CH₃ | H | H | CF₃ | H | H | H | (M + H)⁺ 305.3 |
| | | | | | | | | R_{f} = 0.43 (1/1 |
| | | | | | | | | EtOAc/Hexane) |
| 6 | CH₃ | H | H | 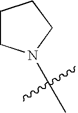 | H | H | H | (M + H)⁺ 306.3 R_{f} = 0.4 (1/1 EtOAc/Hexane) |
| 7 | CH₃ | H | H | C₆H₅ | H | H | H | (M + H)⁺ 313.4 |
| | | | | | | | | R_{f} = 0.6 (1/1 |
| | | | | | | | | EtOAc/Hexane) |
| 8 | CH₃ | Cl | H | H | H | H | H | (M + H)⁺ 271.4 |
| | | | | | | | | R_{f} = 0.65 (1/1 |
| | | | | | | | | EtOAc/Hexane) |
| 9 | CH₃ | F | H | H | H | H | H | (M + H)⁺ 255.2 |
| | | | | | | | | R_{f} = 0.44 (1/1 |
| | | | | | | | | EtOAc/Hexane) |
| 10 | CH₃ | H | H | C₆H₅ | H | H | CH₃ | (M + H)⁺ 251.3 |
| | | | | | | | | R_{f} = 0.38 (2/3 |
| | | | | | | | | EtOAc/Hexane) |
| 11 | CH₃ | H | H | Cl | H | H | 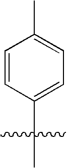 | (M + H)⁺ 361.6 R_{f} = 0.58 (2/3 EtOAc/Hexane) |
| 12 | CH₃ | H | H | 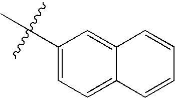 | H | H | H | (M + H)⁺ 287.3 R_{f} = 0.4 (2/3 EtOAc/Hexane) |
| 13 | CF₃ | H | H | F | H | H | H | (M + H)⁺ 309.3 |
| | | | | | | | | R_{f} = 0.66 (2/3 |
| | | | | | | | | EtOAc/Hexane) |
| 14 | CF₃ | H | H | CN | H | H | H | (M + H)⁺ 356.5 |
| | | | | | | | | R_{f} = 0.42 (1/4 |
| | | | | | | | | acetone/hexane) |
| 15 | CH₃ | H | F | H | H | H | H | (M + H)⁺ 255.3 |
| | | | | | | | | R_{f} = 0.32 (2/3 |
| | | | | | | | | EtOAc/Hexane) |
| 16 | CH₃ | H | CN | H | H | H | H | (M + H)⁺ 262.3 |
| | | | | | | | | R_{f} = 0.43 (1/1 |
| | | | | | | | | EtOAc/Hexane) |
| 17 | CF₃ | H | F | H | H | H | H | (M + H)⁺ 349.4 |
| | | | | | | | | R_{f} = 0.55 (2/3 |
| | | | | | | | | EtOAc/Hexane) |
| 18 | CF₃ | H | CN | H | H | H | H | (M + H)⁺ 356.4 |
| | | | | | | | | R_{f} = 0.43 (2/3 |
| | | | | | | | | EtOAc/Hexane) |
| 19 | CF₃ | H | NO₂ | H | H | H | H | (M + H)⁺ 282.1 |
| | | | | | | | | R_{f} = 0.45 (1/1 |
| | | | | | | | | EtOAc/Hexane) |
| 20 | CH₃ | H | NO₂ | Cl | H | H | H | (M + H)⁺ 316.1 |
| | | | | | | | | R_{f} = 0.4 (1/1 |
| | | | | | | | | EtOAc/Hexane) |
| 21 | CH₃ | H | H | 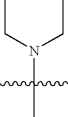 | H | H | H | (M + H)⁺ 308.2 R_{f} = 0.46 (1/1 EtOAc/Hexane) |
| 22 | CH₃ | H | F | F | H | H | H | (M + H)⁺ 273.3 |
| | | | | | | | | R_{f} = 0.41 (2/3 |
| | | | | | | | | EtOAc/Hexane) |
| 23 | CH₃ | H | Br | H | H | H | H | (M + H)⁺ 317.1 |
| | | | | | | | | R_{f} = 0.33 (2/3 |
| | | | | | | | | EtOAc/Hexane) |
| 24 | Et | H | H | F | H | H | H | (M + H)⁺ 269.3 |
| | | | | | | | | R_{f} = 0.29 (2/3 |
| | | | | | | | | EtOAc/Hexane) |
| 25 | 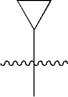 | H | H | F | H | H | H | (M + H)⁺ 281.1 R_{f} = 0.27 (2/3 EtOAc/Hexane) |
| 26 | Et | H | CN | H | H | H | H | (M + H)⁺ 276.2 |
| | | | | | | | | R_{f} = 0.21 (2/3 |
| | | | | | | | | EtOAc/Hexane) |
| 27 | H | H | H | H | H | H | H | (M + H)⁺ 223.1 |
| | | | | | | | | R_{f} = 0.52 (1/1 |
| | | | | | | | | EtOAc/Hexane) |
| 28 | CH₃ | H | H | H | H | H | H | (M + H)⁺ 257.1 |
| | | | | | | | | R_{f} = 0.5 (1/1 |
| | | | | | | | | EtOAc/Hexane) |
| 29 | H | H | H | Cl | H | H | H | (M + H)⁺ 257.1 |
| | | | | | | | | R_{f} = 0.52 (1/1 |
| | | | | | | | | EtOAc/Hexane) |
| 30 | CH₃ | H | H | Cl | H | H | H | (M + H)⁺ 271.2 |
| | | | | | | | | R_{f} = 0.44 (1/1 |
| | | | | | | | | EtOAc/Hexane) |
| 31 | CH₃ | H | OCH₃ | H | H | H | H | (M + H)⁺ 267.2 |
| | | | | | | | | R_{f} = 0.35 (1/1 |
| | | | | | | | | EtOAc/Hexane) |
| 32 | CH₃ | H | H | OCH₃ | H | H | H | (M + H)⁺ 267.2 |
| | | | | | | | | R_{f} = 0.35 (1/1 |
| | | | | | | | | EtOAc/Hexane) |
| 33 | CH₃ | H | H | F | H | H | H | (M + H)⁺ 255.2 |
| | | | | | | | | R_{f} = 0.35 (1/1 |
| | | | | | | | | EtOAc/Hexane) |
| 34 | CH₃ | H | Cl | H | H | H | H | (M + H)⁺ 271.1 |
| | | | | | | | | R_{f} = 0.3 (1/1 |
| | | | | | | | | EtOAc/Hexane) |
| 35 | CO₂ | H | H | F | H | H | H | (M + H)⁺ 299.2 |
| | CH₃ | | | | | | | R_{f} = 0.47 (1/1 |
| | | | | | | | | EtOAc/Hexane) |
| 36 | CH₃ | OMe | H | H | OMe | H | H | (M + H)⁺ 297.3 |
| | | | | | | | | R_{f} = 0.41 (1/1 |
| | | | | | | | | EtOAc/Hexane) |

*NMR data was consistent with the proposed structures

Analytical PLC were obtained using a Gilson HPLC equipped with a quaternary pump, a variable wavelength detector set at 254 nm, a YMC pro C-18 column (50×4.6 mm, 12 μm). The eluents were A: acetonitrile w/0.1% TFA and B: H₂O w/0.1% TFA. Gradient elution from 10% B to 90% over 4 min at a flowrate of 4.0 mL/min was used with an initial hold of 0.5 min and a final hold at 90% B of 0.5 minutes. Total run time was 5 min.

The exemplary compounds 2-36, shown in Table 1 above are named as shown in Table 2

TABLE 2

| Exam- | |
|---|---|
| ple | |
| No. | Compound Name |
| 2 | difluoro{4-[2-(4-methyl(3-pyridyl))(1,3-oxazol-4- |
| | yl)]phenoxy}methane |
| 3 | 4-[2-(4-methyl-3-pyridyl)-1,3-oxazol-4-yl]benzenecarbonitrile |
| 4 | 4-(3,4-dichlorophenyl)-2-(4-methyl(3-pyridyl))-1,3-oxazole |
| 5 | 2-(4-methyl(3-pyridyl))-4-[4-(trifluoromethyl)phenyl]- |
| | 1,3-oxazole |
| 6 | 2-(4-methyl(3-pyridyl))-4-(4-pyrrolidinylphenyl)-1,3-oxazole |
| 7 | 2-(4-methyl(3-pyridyl))-4-(4-phenylphenyl)-1,3-oxazole, 2,2,2- |
| | trifluoroacetic acid |
| 8 | 4-(2-chlorophenyl)-2-(4-methyl(3-pyridyl))-1,3-oxazole |
| 9 | 4-(2-fluorophenyl)-2-(4-methyl(3-pyridyl))-1,3-oxazole |
| 10 | 5-methyl-2-(4-methyl(3-pyridyl))-4-phenyl-1,3-oxazole |
| 11 | 4-(4-chlorophenyl)-2-(4-methyl(3-pyridyl))-5- |
| | (4-methylphenyl)-1,3-oxazole |
| 12 | 2-(4-methyl(3-pyridyl))-4-(2-naphthyl)-1,3-oxazole |
| 13 | 4-(4-fluorophenyl)-2-[4-(trifluoromethyl)(3-pyridyl)]- |
| | 1,3-oxazole |
| 14 | 4-{2-[4-(trifluoromethyl)-3-pyridyl]-1,3-oxazol-4- |
| | yl}benzenecarbonitrile |
| 15 | 4-(3-fluorophenyl)-2-(4-methyl(3-pyridyl))-1,3-oxazole |
| 16 | 3-[2-(4-methyl-3-pyridyl)-1,3-oxazol-4-yl]benzenecarbonitrile |
| 17 | 4-(3-fluorophenyl)-2-[4-(trifluoromethyl)(3-pyridyl)]- |
| | 1,3-oxazole, 2,2,2-trifluoroacetic acid |
| 19 | 2-(4-methyl(3-pyridyl))-4-(3-nitrophenyl)-1,3-oxazole, 2,2,2- |
| | trifluoroacetic acid |
| 20 | 4-(4-chloro-3-nitrophenyl)-2-(4-methyl(3-pyridyl))-1,3-oxazole |
| 21 | diethyl{4-[2-(4-methyl(3-pyridyl))(1,3-oxazol-4- |
| | yl)]phenyl}amine |
| 22 | 4-(3,4-difluorophenyl)-2-(4-methyl(3-pyridyl))-1,3-oxazole |
| 23 | 4-(3-bromophenyl)-2-(4-methyl(3-pyridyl))-1,3-oxazole |
| 24 | 2-(4-ethyl(3-pyridyl))-4-(4-fluorophenyl)-1,3-oxazole |
| 25 | 2-(4-cyclopropyl(3-pyridyl))-4-(4-fluorophenyl)-1,3-oxazole |
| 26 | 3-[2-(4-ethyl-3-pyridyl)-1,3-oxazol-4-yl]benzenecarbonitrile |
| 27 | 4-phenyl-2-(3-pyridyl)-1,3-oxazole |
| 28 | 2-(4-methyl(3-pyridyl))-4-phenyl-1,3-oxazole |
| 29 | 4-(4-chlorophenyl)-2-(3-pyridyl)-1,3-oxazole |
| 30 | 4-(4-chlorophenyl)-2-(4-methyl(3-pyridyl))-1,3-oxazole |
| 31 | 3-methoxy-1-[2-(4-methyl(3-pyridyl))(1,3-oxazol-4-yl)]benzene |
| 32 | 4-methoxy-1-[2-(4-methyl(3-pyridyl))(1,3-oxazol-4-yl)]benzene |
| 33 | 4-(4-fluorophenyl)-2-(4-methyl(3-pyridyl))-1,3-oxazole |
| 34 | 4-(3-chlorophenyl)-2-(4-methyl(3-pyridyl))-1,3-oxazole |
| 35 | methyl 3-[4-(4-fluorophenyl)-1,3-oxazol-2-yl]pyridine-4- |
| | carboxylate |
| 36 | 1,4-dimethoxy-2-[2-(4-methyl(3-pyridyl))(1,3-oxazol-4- |
| | yl)]benzene |

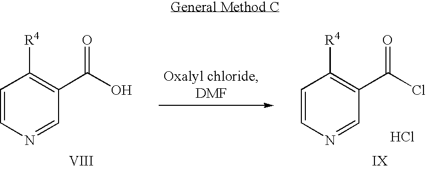

General method C: The nictinic acid chlorides of formula IX are prepared by treating nictinic acids of formula VIII with oxalyl chloride and DMF.

Method C. The syntheses of acid chloride derivatives of the formula Ix are exemplified by the synthesis 4-methyl nicotinic acid chloride, hydrochloride, F.

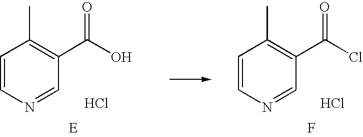

4-methyl nicotinic acid hydrochloride, E (37.5 mmol) was suspended in anhydrous dichloromethane (100 ml) at rt. To this was mixture added oxalyl chloride (20 ml, 2M) slowly, followed by 2 ml anhydrous DMF. The reaction was stirred at room temperature under argon for 2 hours. The solvent was evaporated under reduced pressure and the acid chloride, F, residue was used in the next reaction without further purification.

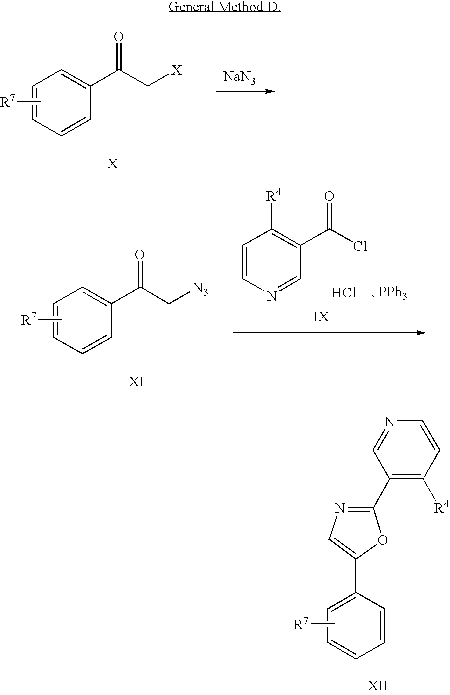

General Method D. The oxazoles of formula XII are prepared by the reaction sequence described in general method D. An alpha-chloro ketone X (generally available commercially) is reacted with NaN₃ and the resulting crude azide of formula XI is reacted with a pyridyl acid chloride IX (prepared in accordance with the scheme for General Method C) and triphenyl phosphine to afford an oxazole of the formula XII.

Method D. The synthesis of compounds of the formula XII are exemplified by the synthesis 3-[5-(4chloro-phenyl)-oxazol-2-yl]-pyridine, example 37.

### EXAMPLE 37

### Preparation of 3-[5-(4-chloro-phenyl)-oxazol-2-yl]-pyridine

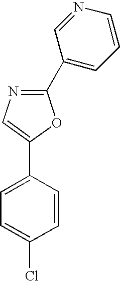

### EXAMPLE 37

### Step 1. Preparation of 2-azido-1-(4-chloro-phenyl)-ethanone, H

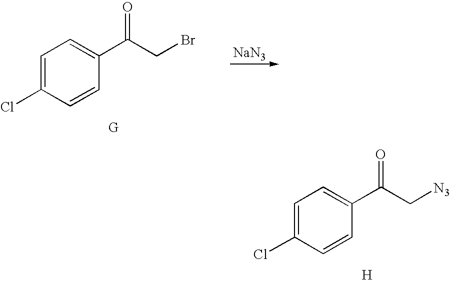

Step 1. A suspension of NaN₃ (0.234 g, 3.6 mmol), 4-chlorophenacyl bromide, G (0.700 g, 3.0 mmol, commercially available) in DMSO (10 ml) was stirred at ambient temperature for 1 hour. The mixture was poured into ice water (100 ml) and was extracted with Et₂O (2×60 ml). The combined organic layers were dried (MgSO₄), and evaporated to crude intermediate, 2-azido-1-(4-chloro-phenyl)-ethanone (0.501 g).

### Step 2. Preparation of 3-[5-(4-chloro-phenyl)-oxazol-2-yl]-pyridine, Example 37

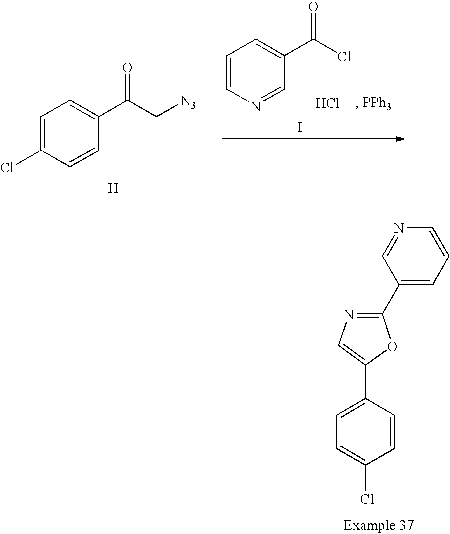

Step 2. Nicotinoyl chloride hydrochloride (0.141 g, 0.79 mmol) was added to a solution of crude intermediate, 2-azido-1-(4-chloro-phenyl)-ethanone (0.155 g, 0.79 mmol), triphenylphosphine (0.355 g, 1.35 mmol) in toluene (20 ml). The resulting suspension was stirred at ambient temperature for 12 hours. The mixture was filtered and the solid was washed with toluene. The filtrates were evaporated and residue was purified by preparative TLC (20×20 cm, 1 mm, 50% EtOAc/hexanes) to afford compound 3-[5-(4-chloro-phenyl)-oxazol-2-yl]-pyridine (27.5 mg, 12%). ¹H-NMR (CD₃OD, 400 MHz) δ 7.55 (d, 2H), 7.88 (s, 1H), 7.90 (d, 2H), 8.25 (m, 1H), 8.95 (d, 1H), 9.24 (d, 1H), 9.55 (s, 1H). LC/MS [M+1]⁺257.09.

TABLE 3
The following exemplary compounds 38-49 of the formula XII, shown in
the scheme General Method D, were prepared according to method D
(example 37) using appropriately modified reagents.

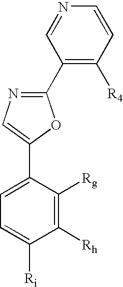

| Entry | R⁴ | R_{g} | Rₕ | Rᵢ | Characterization* |
|---|---|---|---|---|---|
| 38 | CH₃ | H | H | 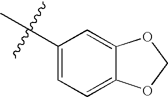 | (M + H)⁺ 273.3 Rₜ = 2.03 min |
| 39 | CH₃ | F | H | F | (M + H)⁺ 273.3 |
| | | | | | Rₜ = 2.1 min |
| 40 | CH₃ | H | F | F | (M + H)⁺ 273.3 |
| | | | | | Rₜ = 2.7 min |
| 41 | CH₃ | Cl | H | H | (M + H)⁺ 271.3 |
| | | | | | Rₜ = 2.52 min |
| 42 | CH₃ | H | F | H | (M + H)⁺ 255.2 |
| | | | | | Rₜ = 2.51 min |
| 43 | CH₃ | H | H | CF₃ | (M + H)⁺ 305.4 |
| | | | | | Rₜ = 2.73 min |
| 44 | CH₃ | Cl | H | CH | (M + H)⁺ 251.4 |
| | | | | | Rₜ = 2.44 min |
| 45 | CH₃ | H | H | OCHF2 | (M + H)⁺ 303.4 |
| | | | | | Rₜ = 2.19 min |
| 46 | CH₃ | H | H | OCH₃ | (M + H)⁺ 267.4 |
| | | | | | Rₜ = 2.04 min |
| 47 | CH₃ | H | H | F | (M + H)⁺ 255.4 |
| | | | | | Rₜ = 2.01 min |
| 48 | CH₃ | H | H | Cl | (M + H)⁺ 361.6 |
| | | | | | R_{f} = 0.58 (2/3 |
| | | | | | EtOAc/Hexane) |
| 49 | H | H | H | Cl | (M + H)⁺ 361.6 |
| | | | | | R_{f} = 0.58 (2/3 |
| | | | | | EtOAc/Hexane) |

*NMR data were consistent with the proposed structures.

For HPLC retention times in table 3: Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 minutes ramped 95% B over 3.5 minutes and held at 95% B for 0.5 minutes and then the column is brought back to initial conditions over 0.1 minutes. Total run time is 4.8 minutes.

The exemplary compounds 38-49 shown in Table 3 are named as shown in Table 4.

TABLE 4

| Example | |
|---|---|
| # | Compound Name |
| 38 | 5-[2-(4-methyl-3-pyridyl)-1,3-oxazol-5-yl]-2H- |
| | benzo[d]1,3-dioxolene |
| 39 | 5-(2,4-difluorophenyl)-2-(4-methyl(3-pyridyl))- |
| | 1,3-oxazole |
| 40 | 5-(3,4-difluorophenyl)-2-(4-methyl(3-pyridyl))- |
| | 1,3-oxazole, chloride |
| 41 | 5-(2-chlorophenyl)-2-(4-methyl(3-pyridyl))- |
| | 1,3-oxazole, chloride |
| 42 | 5-(3-fluorophenyl)-2-(4-methyl(3-pyridyl))- |
| | 1,3-oxazole, chloride |
| 43 | 2-(4-methyl(3-pyridyl))-5-[4-(trifluoromethyl)phenyl]- |
| | 1,3-oxazole, chloride |
| 44 | 2-(4-methyl(3-pyridyl))-5-(4-methylphenyl)- |
| | 1,3-oxazole, chloride |
| 45 | difluoro{4-[2-(4-methyl(3-pyridyl))(1,3-oxazol-5- |
| | yl)]phenoxy}methane, chloride |
| 46 | 4-methoxy-1-[2-(4-methyl(3-pyridyl))(1,3-oxazol-5- |
| | yl)]benzene, chloride |
| 47 | 5-(4-fluorophenyl)-2-(4-methyl(3-pyridyl))-1,3- |
| | oxazole, chloride |

### General Method for Preparing Examples 50-52

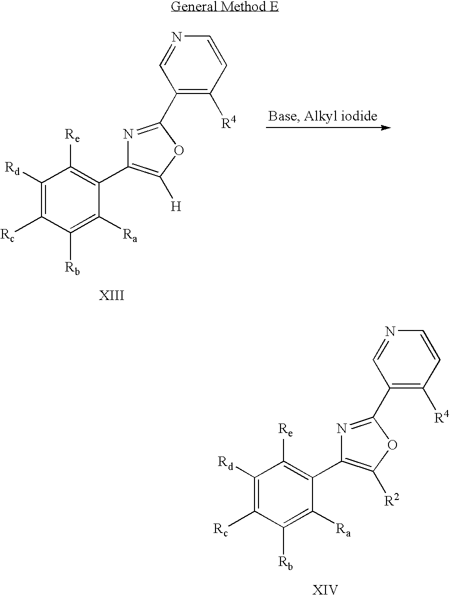

The oxazoles of formula XIV were prepared by the reaction described in general method E. A 3-[4-phenyl)-oxazol-2-yl]-4-methyl-pyridine, XIII (prepared by General Method B) is reacted with a base (e.g. LDA) and an alkyl iodide (generally commercially available) to afford oxazoles of the formula XIV.

Method E. The synthesis of oxazole derivatives of the formula XIV are exemplified by the synthesis of 4-butyl-3-[4-(4-fluorophenyl)-1,3-oxazol-2-yl]pyridine, example 50, 3-[4-(4-fluorophenyl)-5-propyl-1,3-oxazol-2-yl]-4-methylpyridine, example 51, and 3-[4-(4-fluorophenyl)-5-iodo-1,3-oxazol-2-yl]-4-methylpyridine, example 52.

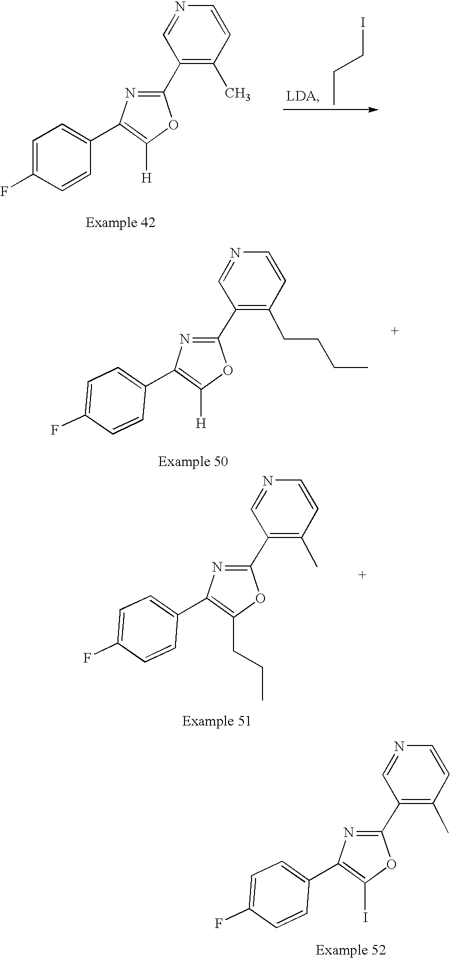

3-[4-(4fluorophenyl)-1,3-oxazol-2-yl]-4methylpyridine example 42 (0.10 g, 0.39 mmol) was dissolved in THF (10.0 mL) and cooled to −78° C. LDA (2.0 M in THF, 0.3 mL, 0.58 mmol) was added at −78° C., and the reaction stirred at −78° C. for 1.5 h. propyliodide (0.66 g, 3.9 mmol) was added and the reaction stirred for additional 1 h at −78° C. The reaction was warmed to rt, and quenched with MeOH. The solvent was then removed, and purification was achieved by preparative TLC (50% EtOAc/Hexanes) afforded 4-butyl-3-[4-(4-fluorophenyl)-1,3-oxazol-2-yl]pyridine, example 50 (11.0 mg) and 3-[4-(4-fluorophenyl)-5-propyl-1,3-oxazol-2-yl]-4-methylpyridine, example 51 (9.0 mg) and 3-[4-(4-fluorophenyl)-5-iodo-1,3-oxazol-2-yl]-4-methylpyridine example 52 (3 mg). LC/MS and NMR were consistent with the assigned structures.

TABLE 5
The following exemplary compounds 50-52 of the formula XII, shown in
the scheme General Method E, were prepared according to method E
(example 50-52) using appropriately modified reagents.

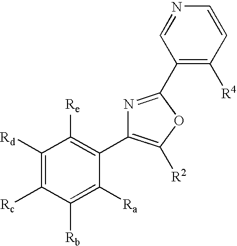

| Entry | R⁴ | Rₐ | R_{b} | R_{c} | R_{d} | Rₑ | R² | Characterization* |
|---|---|---|---|---|---|---|---|---|
| 50 | n-C₄H₉ | H | H | F | H | H | H | (M + H)⁺ 297.1 |
| | | | | | | | | R_{f} = 0.75 (55% |
| | | | | | | | | EtOAc/Hexane) |
| 51 | CH₃ | H | H | F | H | H | n-C₃H₇ | (M + H)⁺ 297.2 |
| | | | | | | | | R_{f} = 0.75 (55% |
| | | | | | | | | EtOAc/Hexane) |
| 52 | CH₃ | H | H | F | H | H | I | (M + H)⁺ 381.1 |
| | | | | | | | | R_{f} = 0.70 (55% |
| | | | | | | | | EtOAc/Hexane) |

*NMR data was consistent with the proposed structures

Analytical HPLC were obtained using a Gilson HPLC equipped with a quaternary pump, a variable wavelength detector set at 254 nm, a YMC pro C-18 column (50×4.6 mm, 12 μm). The eluents were A: acetonitrile w/0.1% TFA and B: H₂O w/0.1% TFA. Gradient elution from 10% B to 90% over 4 min at a flowrate of 4.0 mL/min was used with an initial hold of 0.5 min and a final hold at 90% B of 0.5 minutes. Total run time was 5 min.

The exemplary compounds 50-52 are named as shown in Table 6.

TABLE 6

| Example | |
|---|---|
| No. | Compound Name |
| 50 | 2-(4-butyl(3-pyridyl))-4-(4-fluorophenyl)-1,3-oxazole |
| 51 | 4-(4-fluorophenyl)-2-(4-methyl(3-pyridyl))-5-propyl-1,3- |
| | oxazole |
| 52 | 4-(4-fluorophenyl)-5-iodo-2-(4-methyl(3-pyridyl))-1,3-oxazole |

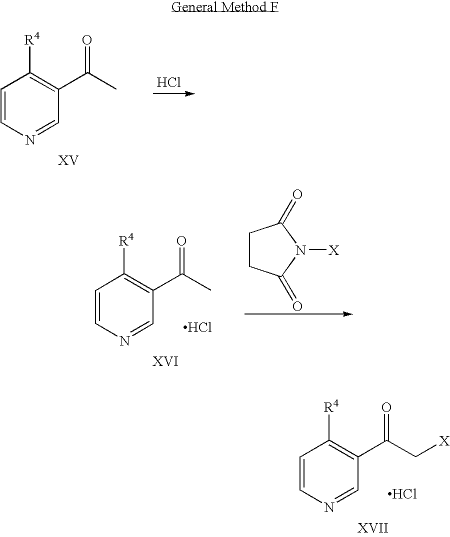

General Method F: The alpha-halo ketones of the formula XVII are prepared by converting ketones, generally commercially available, of the formula XV to the hydrochloride salt of the formula XVI. The hydrochloride salt of the formula XVI is treated with N-halo succinimide to afford the alpha-halo ketones of the formula XVII.

Method F: The syntheses of alpha-halo ketones of the formula XVII are exemplified by the synthesis 2-chloro-1-(3-pyridyl)ethan-1-one, hydrochloride, L.

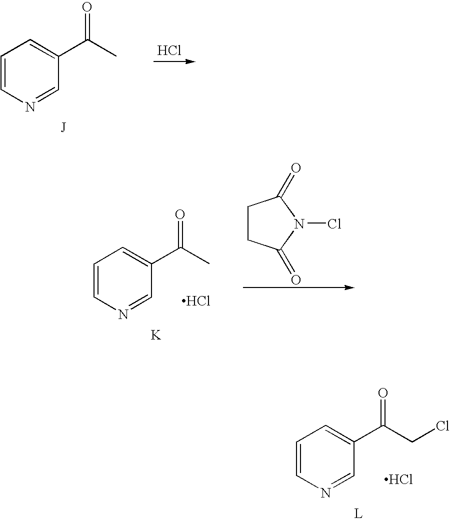

Method F: 3-Acetylpyridine, J (5.0 g, 4.3 mL, 41.3 mmol) was dissolved in ether and the solution was cooled to 0° C. under argon. 2N HCl/ether (1.2 eq, 25 mL) was added and a white solid precipitated. The solid was rinsed with ether and dried, yielding 3-acetyl pyridinium hydrochloride, K, 5.98 g (92%). The 3-acetyl pyridinium hydrochloride, K; was then dissolved in 1 eq of 1N HCl. An equivalent of N-chlorosuccinimide was added and the reaction was heated to refluxing temperature overnight. Ether was added to the reaction mixture and a solid precipitated. The solid was washed with ether and dried under vacuum, providing 3-(2-Chloroacetyl)pyridine hydrochloride H, 6.52 g (83%). The product was used without further purification.

### 2-Chloro-1-(4-methyl-pyridin-3-yl)-ethanone hydrochloride was Prepared Similarly from 3-acetyl-4-methylpyridine

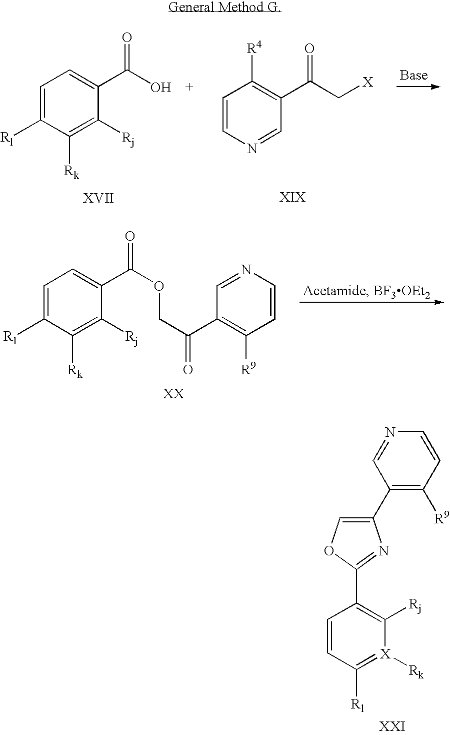

General Method G: The alpha-halo ketones of the formula XIX are reacted with carboxylic acids of the formula XVIII in the presence of base to afford esters of the formula XX. The ester of the formula XX is treated with acetamide and BF₃.OEt₂ to afford the oxazole of the formula XXI.

Method G: The syntheses of oxazoles of the formula XXI are exemplified by the synthesis of 3-[2-(4Chloro-phenyl)-oxazolyl]-4-methyl-pyridine, example 53.

### EXAMPLE 53

### Preparation of 3-[2-(4-Chloro-phenyl)-oxazol4-yl]-4-methyl-pyridine

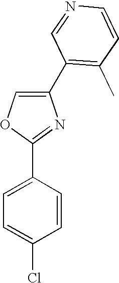

### EXAMPLE 53

### Preparation of of 4-chloro-benzoic acid 2-(4-methyl-pyridin-3-yl)-2-oxo-ethyl ester, O

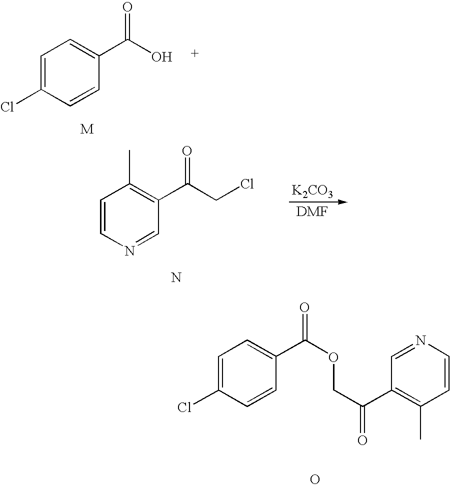

Step 1. 2-chloro-1-(4-methyl-pyridin-3-yl)-ethanone hydrochloride salt, N (1.00 g, 4.88 mmol) was added to a stirred suspension 4-chloro-benzoic acid, M (0.76 g, 4.88 mmol) and potassium carbonate (1.35 g, 9.76 mmol) in N,N-dimethylforamide (60 mL). The resulting mixture was stirred at room temperature for 18 hrs. The reaction was filtered to afford 4-chloro-benzoic acid 2-(4-methyl-pyridin-3-yl)-2-oxo-ethyl ester, O (0.600 g). LC/MS and NMR were consistent with the assigned structures.

### Preparation of 3-[2-(4-Chloro-phenyl)-oxazol4-yl]-4-methyl-pyridine, example 53

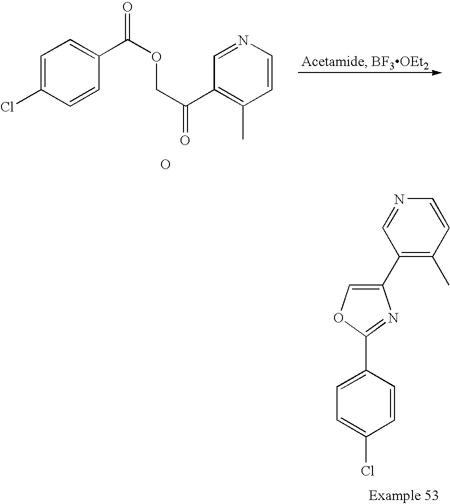

Step 2. To a mixture of 4-chloro-benzoic acid 2-(4-methyl-pyridin-3-yl)-2-oxo-ethyl ester (0.5 g, 1.54 mmol) and acetamide (0.46 g, 7.8 mmol) in p-xylene (20 mL), BF₃ etherate (0.44 mL, 3.1 mmol) was added. The mixture was heated to reflux for 18 hours. After cooling to ambient temperature, NaHCO₃ aqueous was added. The aqueous layer was extracted with EtOAc (3×), and combined organic layers were dried with magnesium sulfate and concentrated. The residue was purified by “PLC to afford 3-[2-(4Chloro-phenyl)-oxazol-4-yl]-4-methyl-pyridine (0.078 g) as a solid. The assigned structure was consistent with the LC/MS and ¹H NMR.

TABLE 7
The following exemplary compounds 54-61 of the formula XXI, shown in
the scheme General Method G, were prepared according to method G
(example 53) using appropriately modified reagents.

| | |
|---|---|
| | XXI |
| 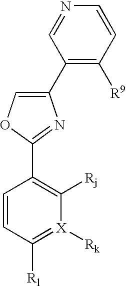 | |

| Entry | R⁹ | Rⱼ | Rₖ | X | Rₗ | Characterization |
|---|---|---|---|---|---|---|
| 54 | CH₃ | H | H | N | — | (M + H)⁺ 297.1 |
| | | | | | | R_{f} = 0.5 (20% |
| | | | | | | MeOH/EtOAc) |
| 55 | CH₃ | H | H | C | OCHF₂ | (M + H)⁺ 303.2 |
| | | | | | | R_{f} = 0.20 (50% |
| | | | | | | EtOAc/Hexane) |
| 56 | CH₃ | H | F | C | F | (M + H)⁺ |
| | | | | | | R_{f} = 0.50 (50% |
| | | | | | | EtOAc/Hexane) |
| 57 | CH₃ | H | H | C | CN | (M + H)⁺ 262.3 |
| | | | | | | R_{f} = 0.21 (50% |
| | | | | | | EtOAc/Hexane) |
| 58 | CH₃ | H | H | C | Cl | (M + H)⁺ 271.4 |
| | | | | | | R_{f} = 0.35 (50% |
| | | | | | | EtOAc/Hexane) |
| 59 | CH₃ | H | H | C | CF₃ | (M + H)⁺ 305.3 |
| | | | | | | R_{f} = 0.35 (70% |
| | | | | | | EtOAc/Hexane) |
| 60 | H | H | H | C | F | (M + H)⁺ |
| | | | | | | R_{f} = 0.2 (50% |
| | | | | | | EtOAc/Hexane) |
| 61 | CH₃ | H | H | C | F | (M + H)⁺ |
| | | | | | | R_{f} = 0.3 (50% |
| | | | | | | EtOAc/Hexane) |

The exemplary compounds 54-61 are named as shown in Table 8.

TABLE 8

| Example | |
|---|---|
| No. | Compound Name |
| 61 | 2-(4-fluorophenyl)-4-(4-methyl(3-pyridyl))-1,3-oxazole |
| 60 | 2-(4-fluorophenyl)-4-(3-pyridyl)-1,3-oxazole |
| 59 | 4-(4-methyl(3-pyridyl))-2-[4-(trifluoromethyl)phenyl]- |
| | 1,3-oxazole |
| 58 | 2-(4-chlorophenyl)-4-(4-methyl(3-pyridyl))-1,3-oxazole |
| 57 | 4-[4-(4-methyl-3-pyridyl)-1,3-oxazol-2- |
| | yl]benzenecarbonitrile, 2,2,2- |
| | trifluoroacetic acid |
| 56 | 2-(3,4-difluorophenyl)-4-(4-methyl(3-pyridyl))-1,3- |
| | oxazole, 2,2,2-trifluoroacetic acid |
| 55 | difluoro{4-[4-(4-methyl(3-pyridyl))(1,3-oxazol-2- |
| | yl)]phenoxy}methane |
| 54 | 4-(4-methyl(3-pyridyl))-2-(3-pyridyl)-1,3-oxazole |

Those skilled in the art will recognize, or be able to ascertain using no more than routine experimentation, many equivalents to the specific embodiments of the invention described herein. Such equivalents are intended to be emcompassed by the following claims.

We claim

1. A compound of formula 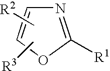
wherein
R¹ represents 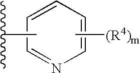
wherein
R⁴ is selected from C₁₋₆ alkyl, C₃₋₅ cycloalkyl, CF₃, and CO₂R⁵, wherein R⁵ is H or C₁₋₄ alkyl; and
m is 0, 1, or 2; or 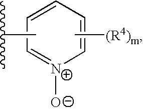
provided that R³ is other than a pyridyl or an N-oxide-containing group; or 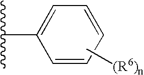
wherein
R⁶ is selected from C₁₋₄ alkyl, CF₃, OCHF₂, CN, NO₂, and halogen; and
n is 0,1, or 2;
R² represents H, C₁₋₆ alkyl, halogen, or tolyl;
R³ represents 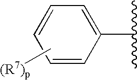
wherein
R⁷ is selected from the group consisting of
C₁₋₄ alkyl,
C₁₋₄ alkoxy,
OCHF₂,
halogen,
CF₃,
CN,
phenyl,
NO₂, 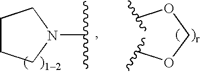
wherein r is 1, 2, or 3, and
N(R⁸)₂ wherein R⁸ is H or C₁₋₄ alkyl, and
p is 0, 1,or2; 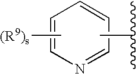
wherein
R⁹ is C₁₋₄ alkyl or C₃₋₅ cycloalkyl, and
s is 0, 1, or 2; or 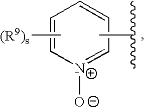
provided that R¹ is other than a pyridyl or an N-oxide-containing group; or 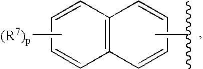
or
C₁₋₄ alkyl; and
one of R¹ and R³ is a 3-pyridyl or 3-pyridyl-N-oxide group which is unsubstituted at the 2- and 6-positions;
or a pharmaceutically acceptable salt thereof.

2. A compound according to claim 1
wherein
R¹ represents 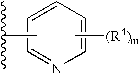
wherein
R⁴ is selected from C₁₋₆ alkyl, C₃₋₅ cycloalkyl, and CF₃; and
m is 0, 1, or 2; or 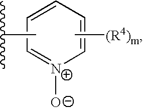
provided that R³ is other than a pyridyl or an N-oxide-containing group; or 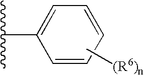
wherein
R⁶ is selected from C₁₋₄ alkyl, CF₃, OCHF₂, and halogen; and
n is 0, 1, or 2;
R³ represents 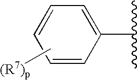
wherein
R⁷ is selected from the group consisting of
C₁₋₄ alkyl,
C₁₋₄ alkoxy,
OCHF₂,
halogen,
CF₃, and 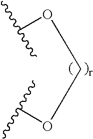
wherein r is 1, 2, or 3, and
p is 0, 1, or 2; and 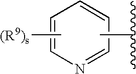
wherein
R⁹ is C₁₋₄ alkyl or C₃₋₅ cycloalkyl; and
s is 0, 1, or 2; or 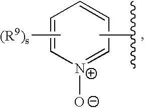
provided that R¹ is other than a pyridyl or an N-oxide-containing group.

3. A compound according to claim 1
wherein
R¹ represents 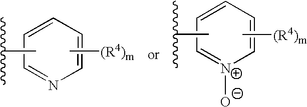
wherein
R⁴ is selected from C₁₋₆ alkyl and C₃₋₅ cycloalkyl; and
m is 0, 1,or 2; or
R² represents H;
R³ represents 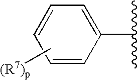
wherein
R⁷ is selected from the group consisting of
C₁₋₄ alkyl,
C₁₋₄ alkoxy,
OCHF₂, and
halogen; and
p is 0, 1, or 2.

4. A compound according to claim 1
wherein
R¹ represents 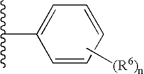
wherein
R⁶ is selected from CF₃, OCHF₂, and halogen; and
n is 0, 1,or 2;
R² represents H; and
R³ represents 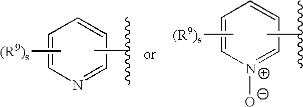
wherein
R⁹ is C₁₋₄ alkyl or C₃₋₅ cycloalkyl and
s is 0, 1, or 2.

5. A pharmaceutical composition comprising a compound of claim 1 and a pharmaceutically acceptable carrier.

6. A method of inhibiting a lyase enzyme, comprising contacting said lyase enzyme with a compound of claim 1.

7. A method of inhibiting a 17α-hydroxylase-C17,20 lyase, comprising contacting a 17α-hydroxylase-C17,20 lyase with a compound of claim 1.

8. A method for treating a subject having a cancer associated with a 17α-hydroxylase-C17,20 lyase, comprising administering to the subject a therapeutically effective amount of a compound of claim 1.

9. A method for treating prostate cancer in a subject, comprising administering to said subject a therapeutically effective amount of a compound of claim 1, such that the prostate cancer in the subject is treated.

10. A method for treating breast cancer in a subject, comprising administering to said subject a therapeutically effective amount of a compound of claim 1, such that the breast cancer in the subject is treated.

11. The method of any one of claims 8-10, wherein said subject is a primate, equine, canine or feline.

12. The method of any one of claims 8-10, wherein said subject is a human.
